(19) United States
(12) Patent Application Publication
(10) Pub. No.: US 2009/0004147 A1
(43) Pub. Date: Jan. 1, 2009

(54) Composition for a healthy gastrointestinal tract

(76) Inventor: Wayne F. Gorsek, Boynton Beach, FL (US)

Correspondence Address: Hoffman, Wasson & Gitler, P.C., Suite 522, 2461 South Clark Street, Arlington, VA, 22202, US

(21) Appl. No.: 11/819,540

(22) Filed: Jun. 28, 2007

(51) Int. Cl. A61K 35/74; A61P 1/00

(52) U.S. Cl. 424/93.4

(57) ABSTRACT

The composition combines Zinc Carnosine; Beta Carotene; Vitamin A; Copper; Calcium Ascorbate; White Tea; Fructo-oligosaccharides; and Probiotic (Acidophilus & Bifidus) to treat, and prevent peptic ulcers and promote a healthy gastrointestinal tract. Each component has an effect on its own combined with a synergistic effect with other components.

### BACKGROUND OF THE INVENTION

Peptic ulcers can cause serious discomfort and damage to the digestive system if left untreated. According to research or other evidence, the following self-care steps may be helpful:
Avoid irritants
Avoid smoking, aspirin and related drugs (nonsteroidal anti-inflammatory drugs), alcohol, coffee (including decaf), and tea to improve ulcer healing
Fight recurrences with fiber
Reduce the risk of new duodenal ulcers by getting enough fiber
Try a little licorice
Take the formulation set forth below
Get a checkup
See a doctor to find out whether your ulcer is related to Helicobacter pylori infection

### SUMMARY OF THE INVENTION

The composition set forth to provide for the prevention and treatment of peptic ulcers. The formulation combines zinc carnosine; beta carotene; Vitamin A; Copper; Calcium Ascorbate; White Tea; Acidophilus & Bifidus; and Fructooligosaccharides to treat, and prevent ulcers and provide a healthy gastrointestinal (GI) tract. Each component has an effect on its own combined with a synergistic effect with other components.

### DETAILED DESCRIPTION OF THE INVENTION

The composition has the following components in the listed ranges:

| | |
|---|---|
| Zinc Carnosine | 10 mg B 500 mg (preferred 50 mg) |
| Beta Carotene | 2,500 IU-250,000 IU (preferred 25,000 IU) |
| Vitamin A | 500 IU-50,000 IU (preferred 5,000 IU) |
| Copper | 0.1-10.0 mg (preferred 1 mg) |
| Calcium Ascorbate | 50 mg-5,000 mg (preferred 500 mg) |
| White Tea | 50 mg-5,000 mg (preferred 500 mg) |
| Fructooligosaccharides | 100 mg-10,000 mg (preferred 1,000 mg) |
| Probiotic (Acidophilus | 50 mg-5,000 mg (preferred 500 mg) |
| and Bifidus) | |

One embodiment of the invention has the following amounts of each component:

| | | |
|---|---|---|
| Zinc Carnosine | 50 | mg |
| Beta Carotene | 25,000 | IU |
| Vitamin A | 5,000 | IU |
| Copper | 1 | mg |
| Calcium Ascorbate | 500 | mg |
| White Tea | 500 | mg |
| Fructooligosaccharides | 1,000 | mg |
| Probiotic (Acidophilus | 500 | mg |
| and Bifidus) | | |

Vitamin A is needed to heal the linings (called mucous membranes) of the stomach and intestines. In one controlled trial, vitamin A supplementation facilitated healing in a small group of people with stomach ulcer.

Zinc is also needed for the repair of damaged tissue and has protected against stomach ulceration in animal studies.

Zinc salt of the amino acid carnosine exerts significant protection against ulcer formation and promotes the healing of existing ulcers.

Acidophilus is a powder form of the normal, friendly bacteria that live in our intestines (called flora). Our intestinal flora helps regulate our digestion and stool patterns.

Beta-carotene is a substance from plants that the body converts into vitamin A. It also acts as an antioxidant and an immune system booster.

Copper is an essential trace element present in the diet and in the human body. It is needed to absorb and utilize iron. It is also part of the antioxidantenzyme, superoxide dismutase (SOD). Copper is needed to make adenosine triphosphate (ATP), the energy the body runs on. Synthesis of some hormones requires copper, as does the synthesis of collagen (the “glue” that holds connective tissue together). In addition, the enzyme, tyrosinase, which plays a role in the production of skin pigment, requires copper to function

Calcium is the most abundant, essential mineral in the human body. Of the two to three pounds of calcium contained in the average body, 99% is located in the bones and teeth. Calcium is needed to form bones and teeth and is also required for blood clotting, transmission of signals in nerve cells, and muscle contraction.

The tea tree grows in Australia and Asia. This tall evergreen tree has a white, spongy bark. The oil from the leaves is used medicinally. White Tea is an essential agreement.

The term “oligosaccharide” refers to a short chain of sugar molecules (“oligo” means “few” and “saccharide” means “sugar.”) Fructo-oligosaccharides (FOS) and inulin, which are found in many vegetables, consist of short chains of fructose molecules. Galacto-oligosaccharides (GOS), which also occur naturally, consist of short chains of galactose molecules. When oligosaccharides are consumed, the undigested portion serves as food for “friendly” bacteria, such as Bifidobacteria and Lactobacillus species.

I claim:

1. A composition to treat, and prevent peptic ulcers and promote a healthy gastrointestinal tract comprising:
Zinc Carnosine; Beta Carotene; Vitamin A; Copper; Calcium Ascorbate; White Tea; Fructooligosaccharides; and Probiotic (Acidophilus and Bifidus).

2. The composition of claim 1, comprising

| | |
|---|---|
| Zinc Carnosine | 10 mg B 500 mg; |
| Beta Carotene | 2,500 IU-250,000 IU; |
| Vitamin A | 500 IU-50,000 IU; |
| Copper | 0.1-10.0 mg; |
| Calcium Ascorbate | 50 mg-5,000 mg; |
| White Tea | 50 mg-5,000 mg; |
| Fructooligosaccharides | 100 mg-10,000 mg; and |
| Probiotic (Acidophilus | 50 mg-5,000 mg. |
| and Bifidus) | |

3. The composition of claim 1, further comprising

| | | |
|---|---|---|
| Zinc Carnosine | 50 | mg; |
| Beta Carotene | 25,000 | IU; |
| Vitamin A | 5,000 | IU; |
| Copper | 1 | mg; |
| Calcium Ascorbate | 500 | mg; |
| White Tea | 500 | mg; |
| Fructooligosaccharides | 1,000 | mg; and |
| Probiotic (Acidophilus | 500 | mg. |
| and Bifidus) | | |
